(19) United States
(12) Patent Application Publication
(10) Pub. No.: US 2025/0270626 A1
(43) Pub. Date: Aug. 28, 2025

(54) USE OF EXONUCLEASE III AND PROBE FOR SENSITIVE AND SPECIFIC LATERAL-FLOW-ASSAY DETECTION OF AMPLIFICATION AMPLICONS

(71) Applicant: GoDx, Inc., Madison, WI (US)

(72) Inventors: Chang Hee KIM, Waunakee, WI (US); Xiao JIANG, Madison, WI (US)

(21) Appl. No.: 18/858,245

(22) PCT Filed: Apr. 18, 2023
(86) PCT No.: PCT/US2023/065902

§ 371 Date: Oct. 18, 2024

Related U.S. Application Data
(60) Provisional application No. 63/332,179, filed on Apr. 18, 2022.

(51) Int. Cl. C12Q 1/6825; C12Q 1/6804; C12Q 1/6844

(52) U.S. Cl. CPC C12Q 1/6825; C12Q 1/6804; C12Q 1/6844

(57) ABSTRACT

The present invention provides methods, kits, and compositions for detecting a target nucleic acid. The methods comprise amplifying the target nucleic acid with a tagged primer, digesting the resulting amplicon with exonuclease III, hybridizing a tagged probe to the digested amplicon, and detecting the resulting dual-tagged amplicon-probe hybrid.

### CROSS-REFERENCE TO RELATED APPLICATIONS

This application claims priority to U.S. Provisional Application No. 63/332,179 filed on Apr. 18, 2022, the contents of which are incorporated by reference in their entireties.

### STATEMENT REGARDING FEDERALLY SPONSORED RESEARCH

This invention was made with government support under grant number R43AI157577 awarded by the National Institute of Allergy and Infectious Diseases of the National Institutes of Health. The government has certain rights in this invention.

### SEQUENCE LISTING

This application includes a sequence listing in XML format titled “165369.00036_ST26.xml”, which is 12,798 bytes in size and was created on Apr. 3, 2023. The sequence listing is electronically submitted with this application via Patent Center and is incorporated herein by reference in its entirety.

### BACKGROUND

Recombinase polymerase amplification (RPA) is an isotheral amplification technique that is popular due to its low incubation temperature (37-42° C.) and its ability to amplify as little as 1-10 copies of a DNA target sequence in less than 20 minutes (US Patent Publication No. 2019/0360030). However, certain aspects of RPA limit its specificity. First, RPA has a high tolerance for mismatches in primers (up to 7 base pairs) and is, thus, more likely to amplify non-target DNA (Mol Cell Probes 29(2):116-21, 2015). Second, RPA requires the use of long primers (ideally over 30 base pairs), which, together with the low incubation temperature, encourages the formation of primer dimers. The resulting “primer noise” is especially severe when low amounts of target DNA are present in the amplification reaction.

To avoid false positives, a true target amplicon can be distinguished from non-target amplicons generated by nonspecific amplification using methods such as gel electrophoresis. However, in low resource or remote settings, the availability of such methods can be limited. Thus, detection of RPA amplicons is commonly performed using a lateral flow assay (LFA). Traditionally, in such assays, one RPA primer comprises a first tag (e.g., an antigen) that specifically binds to a binding agent (e.g., an antibody) immobilized on the nitrocellulose membrane of the LFA strip to form a “test line”. The other RPA primer comprises a second tag that specifically binds to a reporter complex (e.g., a complex comprising gold nanoparticles or colored latex beads). Thus, amplicons containing both tagged primers will bind to both the binding agent immobilized on the LFA strip and the reporter, forming a complex that is visible as a colored band on the test line of the LFA strip. Unfortunately, primer dimers and nonspecific amplicons generated by the two tagged primers produce false-positive results in such assays.

One method for increasing the specificity of RPA assays involves hybridizing an “Nfo probe” to the target amplicon (PLoS Biol 4(7): e204, 2006). The Nfo probe contains a tetrahydrofuran (THF) abasic-site mimic, a first tag (e.g., an antigen) that specifically binds to a binding agent (e.g., an antibody) immobilized on the nitrocellulose membrane of the LFA strip to form a “test line”, and a blocker at its 3′ end to prevent the probe from prematurely acting as a primer. The primer that anneals to the opposite strand contains a second tag that specifically binds to a reporter complex (e.g., a complex comprising gold nanoparticles or colored latex beads) included in the LFA strip. Hybridization of the Nfo probe to complementary DNA enables recognition of the THF by endonuclease IV (Nfo) from Escherichia coli, which specifically digests double-stranded DNA (dsDNA). Cleavage of the probe by Nfo separates the 3′ blocker from the probe. Thus, this cleavage reaction generates a free 3′ OH-end on the 5′ remnant of the incised probe, allowing it to serve as a primer for amplification. The amplicons produced from the cleaved Nfo probe and the primer containing the second tag will form a complex that is visible as a colored band on the test line of the LFA strip. However, while this Nfo probe method increases the specificity of the RPA assay, it is not impervious to false positives. Because the cleaved Nfo probe functions as a primer in this method, this method is susceptible to false positives caused by primer-probe dimers formed at the THF site.

Accordingly, there remains a need in the art for improved methods to validate RPA results in resource-limited settings.

### SUMMARY

In a first aspect, the present invention provides methods for detecting a target nucleic acid. The methods comprise (a) digesting a target amplicon comprising the target nucleic acid and a first 5′ tag with an exonuclease III; (b) hybridizing a probe to a single-stranded portion of the digested amplicon that comprises the first 5′ tag to form an amplicon-probe hybrid, wherein the probe comprises a second 5′ tag and a 3′ blocker; and (c) detecting the amplicon-probe hybrid.

In a second aspect, the present invention provides methods for assaying for the presence of a target nucleic acid in a sample. The methods comprise (a) combining the sample with a forward primer comprising a first 5′ tag, a reverse primer, deoxynucleotide triphosphates (dNTPs), and an amplification master mix to form a reaction mixture, wherein the forward primer and the reverse primer are complementary to the ends of the target nucleic acid; (b) incubating the reaction mixture under conditions suitable for amplification to form an amplification product, wherein the amplification product comprises a target amplicon comprising the target nucleic acid and the first 5′ tag if the target nucleic acid is present in the sample; (c) incubating the amplification product with an exonuclease III to form a digestion product, wherein the digestion product comprises a single-stranded digested target amplicon comprising the first 5′ tag if the target amplicon is present in the amplification product; (d) incubating the digestion product with a probe, wherein the probe hybridizes to the single-stranded digested target amplicon to form an amplicon-probe hybrid if the single-stranded target digested amplicon is present in the digestion product, and wherein the probe comprises a second 5′ tag and a 3′ blocker and is complementary to a portion of the single-stranded digested target amplicon; and (e) assaying for the presence of the amplicon-probe hybrid.

In a third aspect, the present invention provides kits for detecting a target nucleic acid in a sample. The kits comprise (a) a forward primer comprising a first 5′ tag; (b) a reverse primer; (c) a probe comprising a second 5′ tag and a 3′ blocker; and (d) an exonuclease III. To enable amplification of the target nucleic acid sequence, the forward primer and the reverse primer are complementary to the ends of the target nucleic acid. To enable specific detection of the amplified product using the probe, the probe is complementary to an intervening portion of the target nucleic acid.

In a fourth aspect, the present invention provides compositions comprising (a) a forward primer comprising a first 5′ tag and a reverse primer, wherein the forward primer and the reverse primer are complementary to the ends of a target nucleic acid; (b) dNTPs; (c) an amplification master mix; (d) an exonuclease III; and (e) a probe comprising a second 5′ tag and a 3′ blocker, wherein probe is complementary to the target nucleic acid.

### BRIEF DESCRIPTION OF THE DRAWINGS

FIG. 1 is a schematic depiction of the methods for generating a dual-tagged amplicon-probe hybrid for lateral flow assay (LFA) detection described herein.

FIG. 2 is a gel demonstrating the effects of adding exonuclease III to a recombinase polymerase amplification (RPA) reaction. Reaction 1: 200 copies of Neisseria gonorrhoeae (NG) DNA, 250 μM dNTPs, 20-minute incubation. Reaction 2: 200 copies of NG DNA, 250 μM dNTPs, 100 U exonuclease III, 20-minute incubation. Reaction 3: 200 copies of NG DNA, 250 μM dNTPs, 100 U exonuclease III, 40-minute incubation. Reaction 4: 200 copies of NG DNA, 1.8 mM dNTPs, 20-minute incubation. Reaction 5: 200 copies of NG DNA, 1.8 mM dNTPs, 100 U exonuclease III, 20-minute incubation. Reaction 6: 200 copies of NG DNA, 1.8 mM dNTPs, 100 U exonuclease III, 40-minute incubation. The predicted amplicon size is 178 base pairs (bp).

FIG. 3 is a schematic showing where the primers and probe used to detect NG DNA in the examples anneal to an NG target sequence. The arrows indicate where the forward primer (SEQ ID NO: 2), reverse primer (SEQ ID NO: 3), and probe (SEQ ID NO: 4) bind to the target sequence (SEQ ID NO: 1). The arrows point in the 5′ to 3′ direction of the primer or probe. The sequences of these NG primers and probe are provided in Table 1.

FIG. 4 demonstrates that exonuclease III is required for stable amplicon-probe hybridization in an RPA reaction. The top portion of the figure shows LFA results, and the bottom portion of the figure shows gel electrophoresis results. Reactions 1-8 contain 100 U exonuclease III, while reactions 9-16 contain no exonuclease III. Reactions 1 and 9: 1.8 mM dNTPs, 2000 copies NG DNA, 20-minute incubation. Reactions 2 and 10: 1.8 mM dNTPs, non-template control (NTC; i.e., no NG DNA), 20-minute incubation. Reactions 3 and 11: 250 μM dNTPs, 2000 copies of NG DNA, 20-minute incubation. Reactions 4 and 12: 250 μM dNTPs, NTC, 20-minute incubation. Reactions 5 and 13: 1.8 mM dNTPs, 2000 copies of NG DNA, 40-minute incubation. Reactions 6 and 14: 1.8 mM dNTPs, NTC, 40-minute incubation. Reactions 7 and 15: 250 μM dNTPs, 2000 copies of NG DNA, 40-minute incubation. Reactions 8 and 16: 250 μM dNTPs, NTC, 40-minute incubation.

FIG. 5 shows the results of the exonuclease III titration experiment described in Example 3. 200 copies of NG genomic DNA were added to all reactions. Reaction 1: 100 U exonuclease III. Reaction 2: 20 U exonuclease III. Reaction 3: 4 U exonuclease III. Reaction 4: 0.8 U exonuclease III.

FIG. 6 shows the results of adding 20 U (reactions 1-5) and 10 U (reactions 6-10) exonuclease III to the RPA reaction mixture. Reactions 1, 2, 6, and 7: 50 copies of NG genomic DNA. Reactions 3, 4, 8, and 9: 20 copies of NG genomic DNA. Reactions 5 and 10: NTC.

FIG. 7 shows the results of prolonged incubation with 20 U exonuclease III. Each reaction contains 20 copies of NG genomic DNA. Reactions 1 and 2: 20-minute incubation. Reactions 3 and 4: 40-minute incubation. Reactions 5 and 6: 60-minute incubation.

FIG. 8 demonstrates the specificity of LFA detection of the amplicon-probe hybrid. Reactions 1-8: NG primer set with NG probe, 20 U exonuclease III. Reactions 9 and 10: Clostridioides difficile (CDF) primer set with NG probe, 100 U exonuclease III. Reactions 1 and 2: 2000 copies of NG DNA. Reactions 3 and 4: 2000 copies of Trichomonas vaginalis (TV) DNA. Reactions 5 and 6: Chlamydia trachomatis (CT) DNA extract. Reactions 7 and 8: 352 pg human genomic DNA. Reaction 9: 200 copies of CDF DNA. Reaction 10: NTC (water).

FIG. 9 demonstrates successful multiplex LFA detection of Campylobacter (CA) and Salmonella (SA) RPA amplicons. Reactions 1 and 2 show single-plex detection of CA. Reaction 1 contained 3×10⁴ copies of CA genomic DNA, and reaction 2 is a non-template control. Reactions 3 and 4 show single-plex detection of SA. Reaction 3 contains 3×10⁴ copies of SA genomic DNA, and reaction 4 is a negative control. Reactions 5-8 show duplex detection of CA/SA. Reaction 5 contains 3×10⁴ copies of CA genomic DNA, reaction 6 contains 3×10⁴ copies of SA genomic DNA, reaction 7 contains 3×10⁴ copies of both CA and SA genomic DNA, and reaction 8 is the negative control. The expected CA amplicon size is 181 bp and the expected SA amplicon size is 190 bp.

FIG. 10 shows results generated by digesting an RPA amplicon in a separate digestion reaction using various concentrations of exonuclease III. Reactions 1-5 contain 10 U, 1 U, 100 mU, 10 mU, and 1 mU exonuclease III, respectively.

FIG. 11 shows results generated by incubating the separate digestion reactions containing 1 U exonuclease III at 38° C. or room temperature (20° C.) for various lengths of time. Reactions 1-3 were incubated at 38° C. for 5 minutes, 10 minutes, and 20 minutes, respectively. Reactions 4-6 were incubated at room temperature for 5 minutes, 10 minutes, and 20 minutes, respectively.

FIG. 12 is a schematic depiction of how the single-stranded binding protein gp32 protects single-stranded DNA from digestion with exonuclease III. Gp32 binds to the exposed single-stranded region of the amplicon as it is digested by exonuclease III. When exonuclease III encounters a gp32-bound region, digestion is inhibited. This protection prevents the amplicon-probe hybrid from being further digested by exonuclease III.

FIG. 13 shows results generated by including gp32 in the separate digestion reaction. Reactions 1-3 contained 400 ng/μl gp32 and were digested with 1 U exonuclease III for 5 minutes, 20 minutes, and 40 minutes, respectively. Reactions 4-6 included no gp32 and were digested with 1 U exonuclease III for 5 minutes, 20 minutes, and 40 minutes, respectively. Reaction 7 contained 400 ng/μl gp32 and was incubated with NG amplicons and probe at 38° C. for 20 minutes. Reaction 8 contained 400 ng/μl gp32 and 10 U exonuclease III and was incubated at 38° C. for 20 minutes. Reaction 9 contained 400 ng/μl gp32 and 1 U exonuclease III and was incubated with a heterogenous porA probe at 38° C. for 20 minutes.

### DETAILED DESCRIPTION

The present invention provides methods, kits, and compositions for detecting a target nucleic acid. The methods comprise amplifying the target nucleic acid with a tagged primer, digesting the resulting amplicon with exonuclease III, hybridizing a tagged probe to the digested amplicon, and detecting the resulting dual-tagged amplicon-probe hybrid.

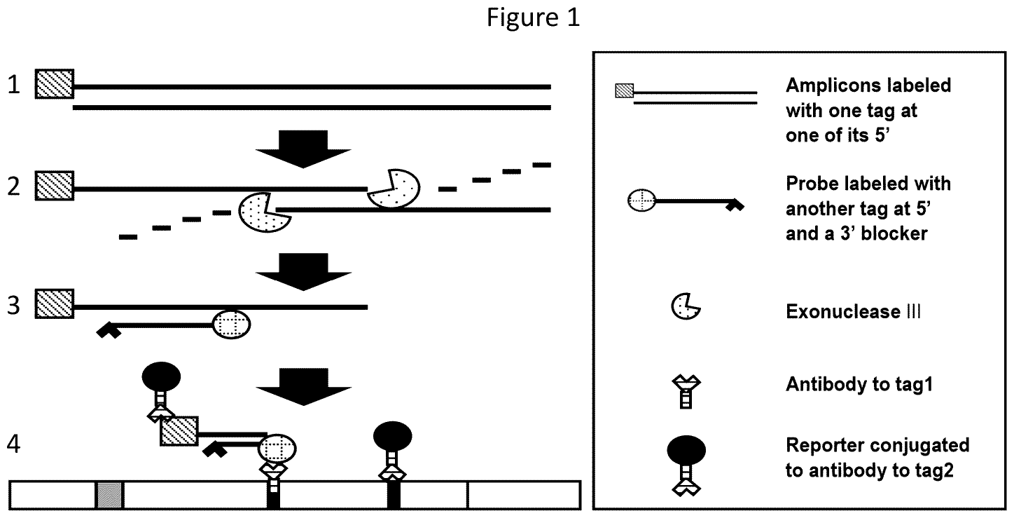

The methods of the present invention are illustrated in FIG. 1. In these methods, an amplification product comprising a target nucleic acid and a 5′ tag (i.e., a target amplicon) is generated via amplification with a tagged forward primer (step 1). The target amplicon is digested with exonuclease III, an enzyme digests the 3′ ends of double-stranded DNA (dsDNA) (step 2). This digestion exposes the single-stranded portion of the target amplicon that is complementary to a probe, allowing the probe to hybridize to the partially digested amplicon (step 3). The probe contains a second tag on its 5′ end and a blocker on its 3′ end. The blocker makes the probe unusable for extension by DNA polymerase and resistant to exonuclease digestion. Hybridization of the digested amplicon to the probe forms an amplicon-probe hybrid comprising a 3′ overhang, which protects the remaining portion of the amplicon from further digestion by exonuclease III. Importantly, digestion of the 3′ end of the amplicon prevents branch migration and prevents new primers from binding to the amplicon and initiating DNA synthesis, both of which would displace the probe. The resulting dual-tagged amplicon-probe hybrid may then be detected using a lateral flow assay (LFA) (step 4).

In some embodiments, the target amplicon is generated using recombinase polymerase amplification (RPA). RPA employs three core enzymes: a recombinase, a single-stranded DNA-binding protein (SSB), and a strand-displacing polymerase. The recombinase is used to pair the primers with homologous sequence in the dsDNA template, the SSB binds to and stabilizes the displaced strands of DNA to prevent the primers from being ejected through branch migration, and the strand displacing polymerase initiates DNA synthesis from the 3′ ends of the bound primers to generate dsDNA amplicons.

RPA is an isothermal amplification method that is performed at a constant temperature (i.e., 37-42° C.) and requires no thermal or chemical melting. Thus, this method can be performed using a simple water bath, heat-block, or a point-of-need device fitted with a heating element. Because this method does not require the use of an expensive thermal cycler instrument, it can be performed in a wider range of settings than conventional polymerase chain reaction (PCR)-based methods. For example, this method can be performed in community hospitals, primary care offices, mobile clinics, and simple physician's office laboratories (POL) that are equipped with a with a basic heater. When this method is incorporated into a point-of-need device, it may also be performed in settings such as homes, nursing homes, workplaces, meat processing plants, prisons, and rapid screening centers without sending samples to a centralized laboratory. Thus, this method is particularly valuable in resource-limited settings in the developing world.

RPA is also high throughput since multiple tests can be set up and run without waiting for instruments to finish running a limited number of samples. The test can be run as needed, without waiting to batch samples for loading into an instrument. Thus, this technology can immediately increase the overall testing capacity of a facility because there is no lead time for purchasing instruments. Further, as is demonstrated in Example 5, the methods can be used for multiplexed detection of multiple target nucleic acids.

### Methods:

In a first aspect, the present invention provides methods for detecting a target nucleic acid. The methods comprise (a) digesting a target amplicon comprising the target nucleic acid and a first 5′ tag with an exonuclease III; (b) hybridizing a probe to a single-stranded portion of the digested amplicon that comprises the first 5′ tag to form an amplicon-probe hybrid, wherein the probe comprises a second 5′ tag and a 3′ blocker; and (c) detecting the amplicon-probe hybrid.

In step (a) of the method, a target amplicon is digested using an exonuclease III. The “target amplicon” is a dsDNA molecule that was generated by amplifying the target nucleic acid. The target amplicon comprises the target nucleic acid and a first 5′ tag. The first 5′ tag may be added to the target amplicon via amplification with a forward primer comprising the first 5′ tag. As used herein, the term “digesting” refers to a process in which an exonuclease III removes nucleotides from the 3′ ends of a dsDNA molecule.

In step (b) of the method, a probe is hybridized to the digested amplicon form an amplicon-probe hybrid. The “amplicon-probe hybrid” comprises the probe hybridized to a single-stranded portion of the digested target amplicon that comprises the first 5′ tag (see step 3 in FIG. 1). Because the probe comprises a second 5′ tag, the amplicon-probe hybrid is “dual-tagged” (i.e., it comprises two tags). As used herein, the term “hybridizing” refers to a process in which a nucleic acid molecule binds to another nucleic acid molecule through the formation of hydrogen bonds between specific nucleotides (i.e., A binds to T or U and G binds to C), forming a double-stranded molecule. Two nucleic acids that have the ability to hybridize to each other in this manner are said to be “complementary”.

In step (c) of the method, the amplicon-probe hybrid is detected. Suitable methods for detecting the amplicon-probe hybrid are described below.

In the methods of the present invention, digestion of the target amplicon by exonuclease III exposes a single-stranded portion of the target amplicon that is complementary to the probe, allowing the probe to hybridize to the partially digested amplicon. Thus, in some embodiments, digestion of the amplicon and hybridization of the probe are performed contemporaneously (i.e., at the same time) in a single vessel. Suitable vessels for use with the present invention include, without limitation, vials, test tubes, microcentrifuge tubes, flasks, bottles, beakers, wells of a multi-well plate, and the like.

In some embodiments, the methods further comprise preparing the target amplicon. In these embodiments, the target amplicon may be prepared by (a) combining a sample comprising the target nucleic acid with a forward primer comprising the first 5′ tag, a reverse primer, deoxynucleotide triphosphates (dNTPs), and an amplification master mix to form a reaction mixture, wherein the forward primer and the reverse primer are complementary to the ends of the target nucleic acid; and (b) incubating the reaction mixture under conditions suitable for amplification to form the target amplicon.

In the Examples, the inventors demonstrate that addition of exonuclease III does not disrupt recombinase polymerase amplification (RPA) reactions. Thus, in some embodiments, incubation of the reaction mixture and digestion of the target amplicon occur contemporaneously in a single vessel. In some embodiments, incubation of the reaction mixture, digestion of the target amplicon, and hybridization of the probe all occur contemporaneously in the vessel.

In the methods described above, the first step involves digesting a target amplicon containing the target nucleic acid. Thus, in this set of methods, the target nucleic acid is always present and is always detected. Accordingly, these methods are referred to as “methods for detecting a target nucleic acid”. However, the methods of the present invention may also be used determine whether the target nucleic acid is present in a sample that may or may not comprise the target nucleic acid. This second set of methods are referred to as “methods for assaying for the presence of a target nucleic acid in a sample”.

Thus, in a second aspect, the present invention provides methods for assaying for the presence of a target nucleic acid in a sample. The methods comprise (a) combining the sample with a forward primer comprising a first 5′ tag, a reverse primer, dNTPs, and an amplification master mix to form a reaction mixture, wherein the forward primer and the reverse primer are complementary to the ends of the target nucleic acid; (b) incubating the reaction mixture under conditions suitable for amplification to form an amplification product, wherein the amplification product comprises a target amplicon comprising the target nucleic acid and the first 5′ tag if the target nucleic acid is present in the sample; (c) incubating the amplification product with an exonuclease III to form a digestion product, wherein the digestion product comprises a single-stranded digested target amplicon comprising the first 5′ tag if the target amplicon is present in the amplification product; (d) incubating the digestion product with a probe, wherein the probe hybridizes to the single-stranded digested target amplicon to form an amplicon-probe hybrid if the single-stranded target digested amplicon is present in the digestion product, and wherein the probe comprises a second 5′ tag and a 3′ blocker and is complementary to a portion of the single-stranded digested target amplicon; and (e) assaying for the presence of the amplicon-probe hybrid.

As is described above, several of these steps (i.e., incubation of the reaction mixture, digestion of the target amplicon, and/or hybridization of the probe) may be performed contemporaneously in a single vessel. Thus, in some embodiments, step (c) and step (d) occur contemporaneously in a single vessel; step (b) and step (c) occur contemporaneously in a single vessel; or step (b), step (c), and step (d) all occur contemporaneously in a single vessel.

As used herein, a “sample” is a substance that comprises or may comprise nucleic acids. The samples used with the present invention may be liquid, solid, or semi-solid. In some embodiments, the sample comprises cells in culture. In other embodiments, the sample is a biological sample obtained from a subject, e.g., a patient. Exemplary patient samples include stool, peripheral blood, sera, plasma, ascites, urine, cerebrospinal fluid (CSF), sputum, saliva, bone marrow, synovial fluid, aqueous humor, amniotic fluid, cerumen, breast milk, bronchoalveolar lavage fluid, semen, prostatic fluid, Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mammary secretions, mucosal secretion, stool, stool water, pancreatic juice, lavage fluids from sinus cavities, bronchopulmonary aspirates, blastocoel cavity fluid, umbilical cord blood, a skin swab sample, a throat swab sample, a genital swab sample, a rectal swab sample, and an anal swab sample. In some embodiments, the sample is an environmental sample. Exemplary environmental samples include soil, rock, plant, water, and air samples. In other embodiments, the sample is a food sample or another consumable. Exemplary food samples include meat, dairy, and produce samples. In some embodiments, the sample comprises nucleic acids that were extracted or purified from a biological material. Methods for extracting and purifying nucleic acids are well known in the art.

A target nucleic acid may be a nucleic acid that one wishes to detect or quantify. In some embodiments, the target nucleic acid is indicative of the presence of a particular organism, such as a pathogen, in a sample. The term “pathogen” refers to an organism that is capable of causing disease in a plant or animal. Exemplary pathogens include bacteria, fungi, viruses, algae, arachnids, insects, nematodes, parasitic plants, and protozoans. In other embodiments, the target nucleic acid is indicative of the presence or absence of a disease or condition. For example, the target nucleic acid may comprise a genetic mutation associated with a disease. In other embodiments, the target nucleic acid is indicative of the prognosis, progression, or response to treatment of a disease or condition. For example, the target nucleic acid may comprise a genetic marker associated with the prognosis of a disease. The target nucleic acid may be derived from genomic DNA (e.g., DNA encoding a protein, open reading frame, or regulatory sequence), mitochondrial DNA, extracellular DNA, plasmid DNA, or cell-free fetal DNA. Alternatively, the target nucleic acid may be derived from an RNA, such as a messenger RNA (mRNA), transfer RNA (tRNA), ribosomal RNA (rRNA), or small nuclear RNA (snRNA). Methods for choosing a target nucleic acid and designing primers to amplify it are known in the art. Many resources, including literature publications and NCBI databases such as “BLAST”, may be used to guide primer design.

In step (a) of the method, reagents are combined to form a reaction mixture. A “reaction mixture” is a mixture of two or more substances that can be used to perform a chemical reaction. The reaction mixture used with the present methods comprise: a forward primer comprising the first 5′ tag, a reverse primer, dNTPs, and an amplification master mix.

A “primer” is a nucleic acid designed to bind via complementary base pairing to the ends of the target nucleic acid. During amplification, DNA polymerases extend primers. A primer's binding site should be unique to the target nucleic acid and have minimal homology to other sequences to ensure specific amplification of the target nucleic acid. “Forward primers” anneal to the antisense strand of double-stranded DNA, which runs in the 3′ to 5′ direction, whereas “reverse primers” anneal to the sense strand of double-stranded DNA, which runs in the 5′ to 3′ direction. In the methods of the present invention, the forward primer comprises a first 5′ tag while the reverse primer is untagged. In some embodiments, the reaction mixture comprises each primer (i.e., a forward primer and reverse primer) at a concentration of 100-1000 nM. In the Examples, either 240 nM of each primer or 480 nM of each primer was utilized in the reaction mixture. Examples of suitable primers for use with the present invention are provided in Tables 1-3 below.

Deoxynucleotide triphosphates (dNTPs) are nucleoside triphosphates containing deoxyribose. They are the building blocks of DNA. In some embodiments, the reaction mixture comprises dNTPs at a concentration of 0.2-2 mM. In the Examples, either 250 μM dNTPs or 1.8 mM dNTPs was utilized in the reaction mixture.

As used herein, the term “amplification master mix” refers to a mixture comprising all the additional reagents (i.e., beyond the forward primer, reverse primer, and dNTPs) needed to perform an amplification reaction. Different reagents may be included in the amplification master mix depending on the selected amplification method, as discussed below.

In some embodiments, the target nucleic acid is RNA, which much be reverse transcribed to allow it to be amplified. Thus, in some embodiments, the reaction mixture further comprises a reverse transcriptase. A “reverse transcriptase” is an RNA-dependent DNA polymerase. It initiates synthesis of a single-stranded complementary DNA (cDNA) using RNA as a template. The resulting cDNA can serve as the input for the amplification reaction. So long as the reverse transcriptase is functional under the amplification conditions (e.g., at 37-42° C. for RPA) the RNA can be reverse transcribed and amplified in a single reaction. The reverse transcription reaction can be performed using the forward or reverse amplification primer to initiate cDNA synthesis. Alternatively, an additional primer may be included in the reaction to serve as the starting point for reverse transcription.

In step (b), the reaction mixture is incubated under conditions suitable for amplification, resulting in the generation of an amplification product (i.e., a product formed by an amplification reaction). If the target nucleic acid is present in the sample, the amplification product will include a target amplicon comprising the target nucleic acid and the first 5′ tag.

The term “conditions suitable for amplification” refers to conditions in which incubation of the reaction mixture will result in the generation of the target amplicon if the target nucleic acid is present in the sample. These conditions include a suitable reaction time, reaction temperature, and concentrations of the various reagents included in the reaction mixture for amplification to occur. The conditions suitable for amplification will depend on the selected amplification method.

The methods of the present invention are designed to be used out in the field or in low resource settings. Thus, in preferred embodiments, an isothermal amplification method is utilized, and incubation of the reaction mixture occurs under isothermal conditions. As used herein, “under isothermal conditions” means that the amplification reaction is conducted at a relatively constant temperature. Suitably, the reaction is conducted with temperature fluctuations less than ±10° C., ±5° C., or ±2° C. In some embodiments, the amplification methods are performed without any equipment requiring a power supply to provide source heat for the amplification reaction. The methods may be performed at a temperature below 70° C., 65° C., 60° C., 55° C., 50° C., 65° C., or 40° C. In certain embodiments, the methods are performed at a temperature below 37° C. Suitably, the methods may be performed at a temperature between 20° C. and 70° C., between 20° C. and 65° C., between 20° C. and 60° C., between 20° C. and 55° C., between 20° C. and 50° C., between 20° C. and 45° C., between 20° C. and 40° C., or between 20° C. and 37° C. For example, in embodiments in which recombinase polymerase amplification (RPA) is utilized, the incubation of the reaction mixture may be performed at 37-40° C.

Suitable isothermal amplification methods for use with the present invention include, without limitation, recombinase polymerase amplification (RPA), loop-mediated isothermal amplification (LAMP), reverse-transcriptase loop-mediated isothermal amplification (RT-LAMP), strand displacement amplification (SDA), nucleic acid sequence-based amplification (NASBA), nicking enzyme amplification reaction (NEAR), transcription-mediated amplification (TMA), and helicase-dependent amplification (HDA). A detailed description of such isothermal amplification methods can be found in Zhao et al. (Chem. Rev 115: 12491-12545, 2015) and in Craw and Balachandran (Lab Chip 12: 2469-2486, 2012).

As is noted above, the amplification master mix comprises all the additional reagents, beyond the primers and dNTPs, necessary to perform the desired amplification method. For example, in embodiments in which the amplification method is RPA, the amplification master mix comprises RPA reagents. The term “RPA reagents” refers to all the reagents necessary to perform RPA. RPA reagents include: a recombinase, a recombination mediator protein, a single-stranded DNA binding protein (SSB), and a stand-displacing DNA polymerase. A detailed description of RPA, RPA reagents, and suitable concentrations of RPA reagents, can be found in U.S. Pat. No. 7,270,981, which is hereby incorporated by reference in its entirety.

A “recombinase” is an enzyme that catalyzes DNA exchange reactions. In RPA, rather than melting the dsDNA in a sample to make primer binding sites accessible as you would in PCR, a recombinase is used to load the primers. The recombinase forms a complex with a primer and scans the dsDNA for a homologous sequence. When the homologous sequence is found, the recombinase performs DNA strand exchange, displacing the complementary strand and allowing the primer to hybridize to the homologous sequence. In some embodiments, the recombinase is UvsX from T4 bacteriophage. In other embodiments, the recombinase is RecA or RecT from E. coli or a homolog thereof.

A “recombination mediator protein” is a protein that regulates homologous recombination (i.e., the exchange of strands between homologous DNA molecules). In some embodiments, the recombination mediator protein is UvsY from T4 bacteriophage. UvsY stimulates the activity of the recombinase UvsX, lowers the critical concentration of UvsX that is required for activity, and promotes strand exchange. In other embodiments, the recombination mediator protein is RecO and/or RecR from E. coli, which stabilize the binding of RecA to single-stranded primers.

A “single-stranded DNA binding protein (SSB)” is a protein that binds to and stabilizes single-stranded DNA (ssDNA). Use of an SSB in the present methods (1) stabilizes the displaced complementary strand so that it does not displace the bound primer and (2) stabilizes the amplicon-probe hybrid against further digestion by exonuclease III. In some embodiments, the SSB is gp32 from T4 bacteriophage.

A “DNA polymerase” is an enzyme that catalyzes the formation of DNA. In embodiments in which the amplification step is performed under isothermal conditions, the DNA polymerase used with the present methods is advantageously a strand-displacing polymerase, i.e. a polymerase with the ability to displace downstream DNA encountered during synthesis. Exemplary strand-displacing DNA polymerases include phi29, Bst, Bsm, Bsu, and Klenow fragment. In some embodiments, the stand-displacing DNA polymerase is the large fragment of Bacillus subtilis polymerase I (Bsu).

In the Examples, the inventors utilized a commercial RPA kit (i.e., the TwistAmp® Liquid Basic Kit from TwistDx) that includes the necessary recombinase, recombination mediator protein, SSB, and stand-displacing DNA polymerase to perform RPA. Thus, in some embodiments, the amplification master mix is provided in the form of a commercial kit.

The amplification master mix may further comprise additional components, including cofactors, buffering agents, crowding agents, amplification enhancers, or any combination thereof. As used herein, a “cofactor” is a substance, other than the substrate, that is essential for the activity of an enzyme. Exemplary cofactors include magnesium, which functions as a cofactor for a variety of polymerases. The cofactor may be introduced to the amplification reaction as a salt, e.g., MgSO₄ or MgCl₂. As used herein, a “buffering agent” comprises a weak acid or base and is used to maintain the acidity (pH) of a solution near a chosen value after the addition of another acid or base. Suitably, the buffering agent may be selected from Tris hydrochloride (Tris HCl), ammonium sulfate ((NH₄)₂SO₄), or potassium chloride (KCl). A “crowding reagent” is an inert, non-charged polymer that is used to occupy space in a reaction. Examples of crowding reagents include polyethylene glycol (PEG), dextran, and Ficoll. As used herein, an “amplification enhancer” is a substance that enhances amplification specificity, efficiency, consistency, and/or yield. Exemplary amplification enhancers include dimethyl sulfoxide (DMSO), glycerol, formamide, polyethylene glycol, N,N,N-trimethylglycine (betaine), bovine serum albumin (BSA), tetramethylammonium chloride (TMAC), a detergent, or combinations thereof. Suitably, the detergent is a nonionic detergent such as Tween 20 or Triton X-100. Examples of suitable concentrations of these components for use in a PCR amplification master mix are as follows: 1-4 mM magnesium (cofactor); 8-12 mM Tris HCl, 40-60 mM (NH₄)₂SO₄, and/or 40-60 mM KCl (buffering agent); 3-10% DMSO, 1.25-10% formamide, 5-6% polyethylene glycol, 0.5-2.5 M betaine, 0.01-0.5 μg/μl BSA, 15-100 mM TMAC, and/or 0.25-1% Tween 20 (amplification enhancer).

With any isothermal amplification technique, the annealase ICP8 and, optionally, a helicase may be included to accelerate the reaction. ICP8 is derived from the herpesvirus DNA replication system. This annealase promotes efficient replication of the viral genome during host cell infection by stabilizing ssDNA and recruiting various factors necessary for replication. Specifically, ICP8 binds ssDNA, samples ssDNA for base pairing, and anneals to ssDNA molecules. Thus, ICP8 can be used to promote the annealing of primers to their complementary targets during an amplification reaction. By increasing reaction kinetics and reducing off-target amplification, ICP8 allows the reaction to be performed at a lower temperature with increased specificity. The ICP8 used with the present invention may be from any available source, including from any herpesvirus or another closely related virus. For instance, the ICP8 may be derived from chelonid herpesvirus 5, a type of herpesvirus that infects the Hawaiian green sea turtle, which has an internal body temperature of 20-25° C. Since ICP8 lacks helicase function, helicases or nucleases are added to the reaction to generate ssDNA for ICP8 to sample. Any suitable helicase or nuclease may be used with the methods of the present invention. Exemplary helicases include, without limitation, UvrD, RecBCD, BLM, WRN, and RecQ. Exemplary nucleases include UL12, nickases, and restriction enzymes.

The largest limitation to the selection of enzymes for use in the amplification reaction is their ability to function at the temperature at which the reaction is performed. However, it is standard practice for one of skill in the art to optimize reaction conditions and enzyme components to achieve particular reaction goals (i.e., sensitivity, specificity, speed, and efficiency at a given temperature). For instance, conditions such as primer length, melting temperature (Tm), GC content, reaction buffering conditions (e.g., pH, salt concentrations, dNTP concentrations), and crowding agents (e.g., PEG) can be varied. Literature regarding isothermal amplification reactions and ICP8 DNA binding, strand-invasion, and recombination assays may provide guidelines that aid in reaction optimization.

In step (c), the amplification product is digested with exonuclease III to form a digestion product (i.e., a product formed by a digestion reaction). If the target nucleic acid is present in the sample, the amplification product will include the target amplicon, and the digestion product will include a single-stranded digested target amplicon comprising the first 5′ tag.

Exonuclease III is a dsDNA-specific exonuclease that catalyzes the stepwise removal of nucleotides from 3′-hydroxyl termini. Exonuclease III removes a limited number of nucleotides during each binding event, resulting in the progressive degradation of DNA molecules. Notably, exonuclease III activity is inhibited by 3′ overhangs that are at least four bases in length. In some embodiments, the exonuclease III is from E. coli. However, E. coli exonuclease III is inactivated by temperatures higher than 70° C. Thus, for amplification reactions that are performed at higher temperatures (e.g., LAMP, which is performed at 65° C.), a thermostable exonuclease III should be utilized. One example of a thermostable exonuclease III is the exonuclease III from Sulfolobus sp. Tu B-1 (www.uniprot.org/uniprot/B5MF73). In some embodiments, 1-100U exonuclease III is used in the reaction. In some embodiments, 1 U exonuclease III is used. In other embodiments, 10-20 U exonuclease III is used.

In step (d), the probe is hybridized to the single-stranded digested target amplicon if it is present in the digestion product, forming an amplicon-probe hybrid. The “probe” used with the present invention is a single-stranded oligonucleotide that comprises a second 5′ tag and a 3′ blocker. The probe may be about 15-40 nucleotides in length. In some embodiments, the reaction mixture comprises the probe at a concentration of 100-1000 nM. In the Examples, either 240 nM probe or 400 nM probe was utilized in the reaction mixture. As used herein, the term “3′ blocker” refers to a moiety that is attached to the 3′ end of the probe to prevent the extension of the probe by DNA polymerase and make it resistant to exonuclease digestion. Suitable 3′ blockers include phosphorothioate bonds, 3′-inverted thymidine (dT), 5′ inverted dT, and sugar moieties (e.g., ribo, 2′-methoxy and 2′-methoxyethyl groups). In the examples, the inventors used an inverted dT as a 3′ blocker. Thus, in some embodiments, the 3′ blocker is an inverted base.

In step (e), an assay is performed to determine whether the amplicon-probe hybrid is present. Amplification using a tagged forward primer and hybridization of a tagged probe generates a dual-tagged amplicon-probe hybrid for detection. The amplicon-probe hybrid may be assayed for using any suitable method including, without limitation, a binding assay, a colorimetric assay, an electrophoretic assay, a fluorescence assay, a turbidity assay, an electrochemical assay, and the like.

In some embodiments, the amplicon-probe hybrid is detected by binding the amplicon-probe hybrid to a first binding agent immobilized on a substrate (i.e., a surface) and to a second binding agent conjugated to (i.e., joined together with) a detectable label. In these embodiments, the first binding agent specifically binds to either the first 5′ tag or the second 5′ tag and the second binding agent specifically binds to the other 5′ tag such that the first binding agent, the amplicon-probe hybrid, the second binding agent, and the detectable label form a sandwich-like complex (see step 4 in FIG. 1) that can be detected via the detectable label.

As used herein, a “5′ tag” is a moiety that is conjugated to the 5′ end of a nucleic acid that specifically binds to a binding agent. The “binding agent” may be any moiety that specifically binds to the 5′ tag. Suitable 5′ tag-binding agent pairs include, without limitation, ligand-protein pairs, antibody-antigen pairs, and antibody-hapten pairs.

In the Examples, the inventors used a forward primer comprising a fluorescein isothiocyanate (FITC) 5′ tag to amplify the target nucleic acid and used an anti-FITC antibody as the first binding agent. The anti-FITC antibody was immobilized to the LFA strip such that binding of the FITC 5′ tag to the anti-FITC antibody captured the amplicon-probe hybrid on the test line of the LFA strip. The inventors hybridized a probe comprising a biotin 5′ tag to the digested target amplicon and used streptavidin as the second binding agent. The streptavidin was conjugated to a detectable label such that binding of the biotin 5′ tag to streptavidin conjugated the amplicon-probe hybrid to a detectable label. Thus, in some embodiments, first 5′ tag is a FITC tag, the first binding agent is an anti-FITC antibody, the second 5′ tag is a biotin tag, and the second binding agent is streptavidin. However, either the first 5′ tag (i.e., the tag on the target amplicon) or the second 5′ tag (i.e., the tag on the probe) may be conjugated to the LFA strip so long as the other 5′ tag is conjugated to the detectable label. Thus, in some embodiments, either the first 5′ tag or the second 5′ tag is FITC, and either the first binding agent or the second binding agent is an anti-FITC antibody, and, in some embodiments, either the first 5′ tag or the second 5′ tag is biotin, and either the first binding agent or the second binding agent is streptavidin.

A “detectable label” is a moiety that can be linked to a molecule of interest to make it detectable. Exemplary detectable labels include fluorescent labels, chemiluminescent labels, colorimetric labels, gold nanoparticles, and quantum dots. For instance, in the LFAs described in the Examples, colored beads that form a visible line when they are conjugated to the test line were used as the detectable label.

### Kits:

In a third aspect, the present invention provides kits for detecting a target nucleic acid in a sample. The kits comprise (a) a forward primer comprising a first 5′ tag; (b) a reverse primer; (c) a probe comprising a second 5′ tag and a 3′ blocker; and (d) an exonuclease III. To enable amplification of the target nucleic acid sequence, the forward primer and the reverse primer are complementary to the ends of the target nucleic acid. To enable specific detection of the amplified product using the probe, the probe is complementary to an intervening portion of the target nucleic acid.

In some embodiments, the kits further comprise additional reagents such as dNTPs and/or an amplification master mix.

In some embodiments, the kits further comprise a detection apparatus. Any detection apparatus that provides a readout that indicates whether a target nucleic acid is present in a sample may be used with the present invention. Detection devices may provide an analog or digital readout.

In the Examples, the inventors used a lateral flow assay (LFA) to detect the amplicon-probe hybrid. Thus, in some embodiments, the detection apparatus is a lateral flow device. As used herein, a “lateral flow device” is a porous device capable of detecting the presence of a target nucleic acid traversing one or more beds. Lateral flow devices typically comprise (a) a sample loading area at one end; (b) an area comprising a capture agent comprising a first binding agent immobilized on a substrate; (c) an area comprising a reporter comprising a detectable label conjugated to a second binding agent, wherein the reporter is not bound to the lateral flow device and is capable of wicking across the lateral flow device; and (d) absorbent material, wherein the absorbent material wicks an aqueous sample across the lateral flow device when the aqueous sample is added to the sample loading area. Thus, in some embodiments, the lateral flow device comprises a sample loading area, an amplification area, a solid support, an absorbent sample pad, or any combination thereof. A detailed description of exemplary lateral flow devices can be found in U.S. Patent Publication No. 2018/0148774, which is hereby incorporated by reference.

In embodiments that utilize a lateral flow device, the assay results may be displayed using LFA strips. The strips comprise a capture agent comprising a first binding agent immobilized on the lateral flow device in a region referred to as the “test area”. The test area can be any shape with well-defined boundaries, such as a dot or a line. The first binding agent binds to the either the first or second 5′ tag of the amplicon-probe hybrid, capturing it in the test area. The first binding agent may be immobilized on the lateral flow device by covalent coupling or affinity binding. The LFA strip may comprise multiple test areas that are designed to capture different target nucleic acids for multiplex detection. Binding of the reporter to the other 5′ tag enables detection of the bound amplicon-probe hybrid via the detectable label.

In some embodiments, the detection device is configured such that detection is accomplished by visual inspection, either with or without additional instrumentation. For example, results can be quantified by imaging and analysis with a computer. The results can be scanned with a smartphone and electronically sent to a clinician, e.g., using an Adobe Acrobat grayscale converter or an Image J image processing software to quantify the visible light signal from a gold nanoparticle. Likewise, a color wheel for visualization of positive tests may be utilized.

In some embodiments, the devices further comprise a heating element, e.g., a heating element is portable and does not require electricity. The heating element may comprise a battery-powered, cell phone-powered, or solar battery-powered heating film. Alternatively, the heating element may use a reversible or irreversible exothermic chemical reaction to generate heat.

The kits may also include primers and reagents for detecting a control nucleic acid, i.e., a nucleic acid other than the target nucleic acid. Detection of the control nucleic acid may indicate that the method is working (i.e., a positive control) or may indicate that the method is producing non-specific results (i.e., a negative control).

### Compositions:

In a fourth aspect, the present invention provides compositions comprising (a) a forward primer comprising a first 5′ tag and a reverse primer, wherein the forward primer and the reverse primer are complementary to the ends of a target nucleic acid; (b) dNTPs; (c) an amplification master mix; (d) an exonuclease III; and (e) a probe comprising a second 5′ tag and a 3′ blocker, wherein probe is complementary to the target nucleic acid.

These compositions can be used in a method for assaying for the presence of a target nucleic acid in a sample. These methods comprise: (a) combining the sample with the composition to form a reaction mixture; (b) incubating the reaction mixture under conditions suitable for amplification to form a reaction product, wherein the reaction product comprises an amplicon-probe hybrid if the target nucleic acid is present in the sample, and wherein the amplicon-probe hybrid comprises the probe hybridized to a portion of a single-stranded digest of an amplicon of the target nucleic acid (i.e., a target amplicon) comprising the first 5′ tag; and (c) assaying for the presence of the amplicon-probe hybrid.

In some embodiments, the compositions further comprise: (f) the target nucleic acid; (g) an amplicon of the target nucleic acid comprising the first 5′ tag; and (h) an amplicon-probe hybrid, wherein the amplicon-probe hybrid comprises the probe hybridized to a portion of a single-stranded digest of the target amplicon comprising the first 5′ tag.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: Addition of Exonuclease III does not Inhibit Recombinase Polymerase Amplification (RPA)

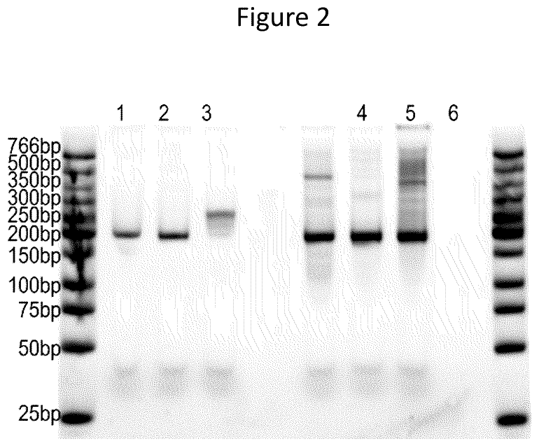

Exonuclease III from E coli was purchased from New England Biolabs (NEB, Ipswich, MA, USA) and a TwistAmp® Liquid Basic Kit was purchased from TwistDx (Maidenhead, UK). RPA reaction mixtures with a 25 μL total reaction volume were prepared following the manufacturer's instructions. Each reaction mixture contained: 100 U exonuclease III, 250 μM or 1.8 mM dNTPs, 480 nM forward primer (see Table 1 for primer sequences), 480 nM reverse primer, and 200 copies of genomic DNA from Neisseria gonorrhoeae (NG) (700825DQ™, ATCC, Manassas, VA). The reaction mixtures were incubated at 38° C. for 20 minutes or 40 minutes. The resulting amplicons were cleaned up using the Monarch® PCR & DNA Cleanup Kit (NEB) and analyzed via 8% polyacrylamide gel electrophoresis. The results of this analysis suggest that the inclusion of 100 U exonuclease III does not affect amplification when (a) 1.8 mM dNTPs is used and the reaction is incubated for up to 40 minutes or (b) 250 μM dNTPs are used and the reaction is incubated for up to 20 minutes (FIG. 2).

### Example 2: Addition of Exonuclease III is Necessary for Stable Amplicon-Probe Hybridization

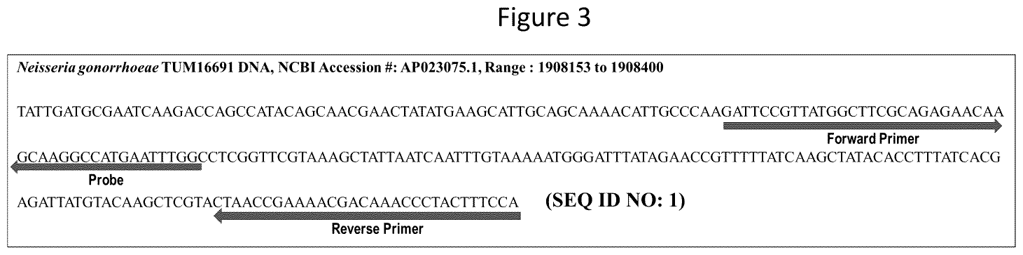

To detect the amplicon-probe hybrid generated by amplification with the primers and probe listed in Table 1 and depicted in FIG. 3 (which are designed to detect the NG damH gene), 2 μL of RPA reaction mixture was loaded onto the sample pad of a lateral flow assay (LFA) strip, which was prepared in house, followed by 2 drops of a running buffer. The LFA strip comprises streptavidin-conjugated 400 nm latex beads on its conjugation pad and 0.5 mg/ml anti-FTIC antibody stripped onto its test line. The control line contains a biotin-IgG conjugate.

TABLE 1
Primers and probe for detection of Neisseria
gonorrhoeae (NG)

| Oligo | Sequence (5′-3′) |
|---|---|
| Forward | /56-FITC/GATTCCGTTATGGCTTCGCAGAGAACAA |
| primer | (SEQ ID NO: 2) |
| Reverse | TGGAAAGTAGGGTTTGTCGTTTTCGGTTAG |
| primer | (SEQ ID NO: 3) |
| Probe | /5Biosg/CCAAATTCATGGCCTTGC/3InvdT/ |
| | (SEQ ID NO: 4) |

*56-FITC: 5′ FITC tag, 5Biosg: 5′ biotin tag, 3InvdT: 3′ inverted thymine blocker

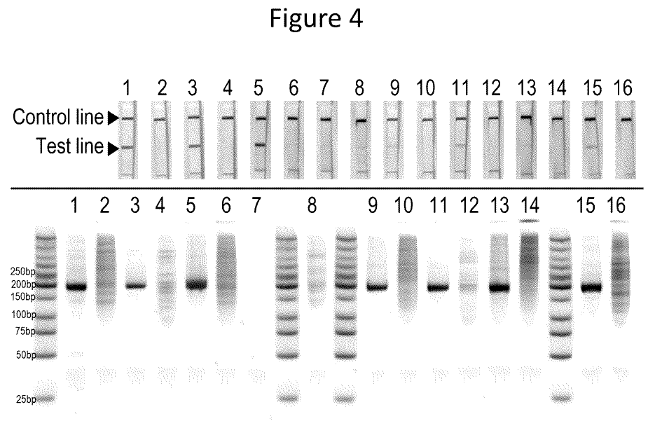

The results of both LFAs and polyacrylamide gel electrophoresis are shown in FIG. 4. In the absence of exonuclease III, the test line is faint as most of the amplicons remain double-stranded such that the hybridized probe can be displaced by Bsu polymerase or by branch migration. In the presence of 100 U exonuclease III, RPA reactions containing either 250 μM dNTPs or 1.8 mM dNTPs resulted in a strong test line following a 20-minute incubation. However, the test line disappeared after a 40-minute incubation when 250 μM dNTPs were used. This suggests that either (1) Bsu polymerase has a slower amplification rate when dNTP concentrations are low, or (2) the dNTPs are depleted before the end of the 40-minute incubation, allowing the exonuclease to fully digest the double-stranded amplicons. As a result, it may be preferable to use 1.8 mM dNTPs and a lower concentration of exonuclease III in the RPA reaction.

### Example 3: Use of 10-20 U Exonuclease III Enables Sensitive LFA Detection of the Amplicon-Probe Hybrid

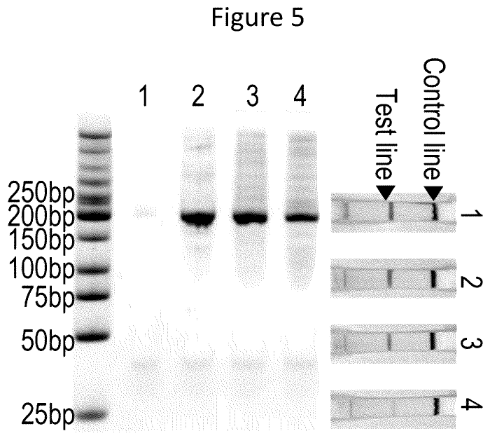

A series of three 1:5 dilutions of 100 U exonuclease III were added to 25 μL RPA reaction mixtures prepared as described in Example 2. 200 copies of NG genomic DNA, 1.8 mM dNTPs, 480 nM of each of the forward and reverse primer, and 240 nM probe were used. The reaction mixtures were incubated at 38° C. for 20 minutes. The results of both LFAs and polyacrylamide gel electrophoresis are shown in FIG. 5. These results demonstrate that 20 U exonuclease III is adequate to generate enough amplicon-probe hybrid to generate a strong test line signal.

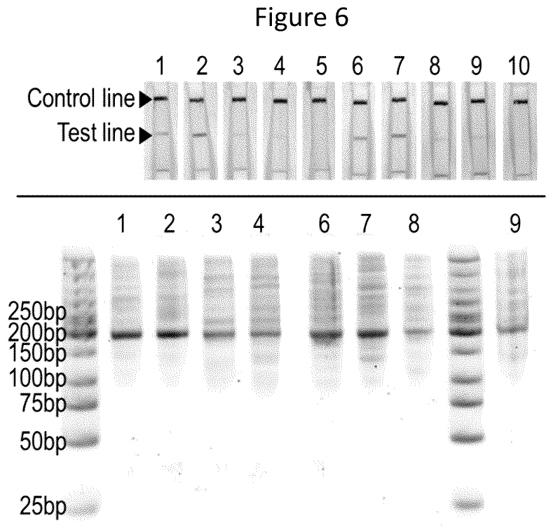

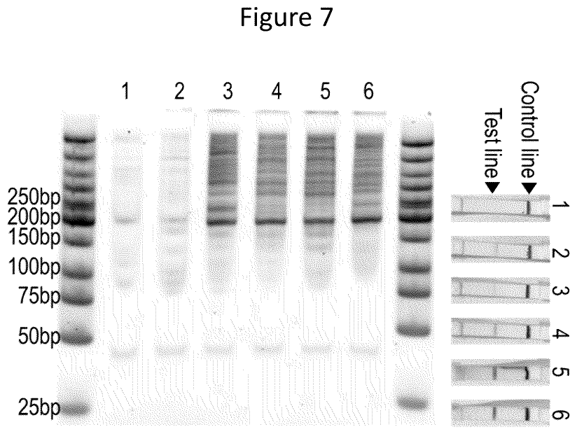

Further experiments were conducted to compare the effect of using 20 U and 10 U exonuclease III for the detection of 50 copies and 20 copies of target NG DNA. Both exonuclease III concentrations resulted in strong test line signals for 50 copies of target DNA and faint test line signals for 20 copies of target DNA in a LFA after a 20-minute incubation (FIG. 6). Next, the effects of prolonged incubations (i.e., 40 minutes and 60 minutes) were tested. The results of this analysis showed that a strong test line signal can be achieved with 20 copies (i.e., 0.8 copies/μL) of target NG DNA after a 60-minute incubation (FIG. 7).

### Example 4: LFA Detection of the Amplicon-Probe Hybrid is Specific

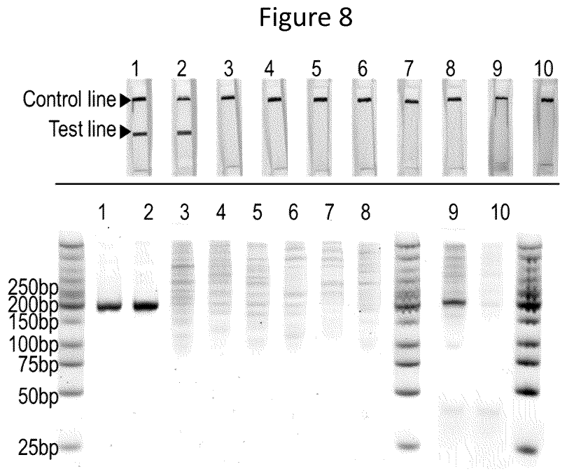

The specificity of the NG primers/probe set was tested against Trichomonas vaginalis (TV) genomic DNA (30001DQ™, ATCC), Chlamydia trachomatis (CT) DNA extracted from a control material (C. trachomatis-High, strain D-UW3, NATCT/NGP-C, ZeptoMetrix, Buffalo, NY), and human genomic DNA (G3041, Promega, Madison, WI). RPA reaction mixtures were prepared as described in Example 2 with 20 U exonuclease III and 1.8 mM dNTPs. The LFA results showed no false positives, even when non-specific DNA products were observed through gel electrophoresis (FIG. 8). The specificity of the NG probe alone was tested by using it with a primer set designed to amplify a Clostridioides difficile (CDF) target sequence. A forward primer tagged with FTIC and a reverse primer (see Table 2 for primer sequences) were used to amplify 200 copies of CDF genomic DNA (BAA-1382DQ™, ATCC) via RPA in the presence of exonuclease III. The CDF primer set amplifies a product that is 172 bp in size. As is shown in FIG. 8, the NG probe did not hybridize with the amplicon produced using the CDF primer set in the presence of 100 U exonuclease III. Together, these results suggest that the detection method described herein has high specificity.

TABLE 2
Primers for amplification of Clostridioides
difficile (CDF)

| Oligo | Sequence (5′-3′) |
|---|---|
| Forward | /56-FAM/CTATAGTTGAATCTTCTACCACTGAAGCA |
| primer | (SEQ ID NO: 5) |
| Reverse | GTAGGTACTGTAGGTTTATTGATTTGAGA |
| primer | (SEQ ID NO: 6) |

*56-FAM: 5′ FAM tag

### Example 5: Multiplexed LFA Detection

To determine whether the detection assay described herein could be performed in a multiplexed manner, multiplexed LFA strips were made in house. The multiplexed LFA strips contained two test lines: a first test line containing an anti-TAMR antibody, and a second test line containing an anti-FITC antibody. Primers and probes for the specific detection of Campylobacter (CA) and Salmonella (SA) were designed for use in a one-pot duplex RPA reaction mixture containing exonuclease III. The sequences of the primers and probes are listed in Table 3, below.

TABLE 3
Primers and probes for multiplexed detection
of Campylobacter (CA) and Salmonella (SA)

| Oligo | Sequence (5′-3′) |
|---|---|
| CA- | /56-TAMRA/GTGGTGCTAAGGCAATGATAGAAGATGGA |
| Forward | (SEQ ID NO: 7) |
| primer | |
| CA- | GCACTTCCATGACCACCTCTTCCAATAACTTC |
| Reverse | (SEQ ID NO: 8) |
| primer | |
| CA- | /5Biosg/GGCATATTGTGCCATCCAAA/3InvdT/ |
| Probe | (SEQ ID NO: 9) |
| SA- | /56-FAM/ATCAACCAGATAGGTAGGTAATGGAATGAC |
| Forward | (SEQ ID NO: 10) |
| primer | |
| SA- | CTACAAGCATGAAATGGCAGAACAGCGT |
| Reverse | (SEQ ID NO: 11) |
| primer | |
| SA- | /5Biosg/GGTACTAATGGTGATGATCA/3InvdT/ |
| Probe | (SEQ ID NO: 12) |

*56-TAMN: 5′ TAMN tag, 5Biosg: 5′ biotin tag, 3InvdT: 3′ inverted thymine blocker, 56-FAM: 5′ FAM tag

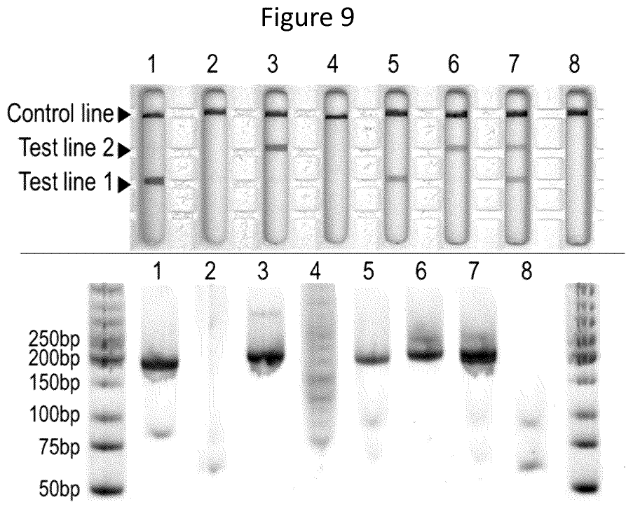

The CA and SA primer and probe sets were first validated by performing single-plex reactions separately, as described above, with a 25 μL total reaction volume. Genomic DNA extracted from Campylobacter jejuni Z086 (ZeptoMetrix, 0801650) or Salmonella enterica Serovar Typhimurium Z005 (ZeptoMetrix, 0801437) bacteria was used as the target DNA. The extracted DNA was quantified using a Qubit 4 Fluorometer, and the genome copy numbers were estimated based on the genome size of the bacteria. To perform a duplex reaction, a total reaction volume of 50 μl was used and the concentrations of exonuclease III, dNTPs, and the TwistAmp® Liquid Basic Kit components were kept the same as in the single-plex 25 μL reaction. The duplex reaction contained 240 nM of each of the four primers (i.e., CA-Forward, CA-Reverse, SA-Forward, and SA-Reverse), 240 nM of each probe (i.e., CA-Probe and SA-Probe), and 3×10⁴ copies of each bacterial genome. The results of this experiment indicate that both bacterial target DNA sequences were successfully amplified in the duplex reaction, and that LFA detection successfully distinguished the two amplicons (FIG. 9).

### Example 6: Optimization of the Exonuclease III Digestion

In the following example, the inventors demonstrate that exonuclease III digestion can be performed on a purified dsDNA amplicon and they identify suitable conditions (i.e., quantity of exonuclease III, digestion time and temperature, and inclusion of gp23) for this separate digestion step. Their results demonstrate that amplicons generated by nucleic acid amplification methods other than RPA can be incubated with exonuclease III and a tagged probe for detection via LFA.

First, the NG primer and probe set (FIG. 3) was used to generate a tagged amplicon (178 bp) using the RPA TwistAmp® Liquid Basic Kit without exonuclease III. The resulting amplicon was purified using the Monarch® PCR & DNA Cleanup Kit and was then quantified using a Qubit 4 Fluorometer. 65 ng of amplicon were used for the experiments described below.

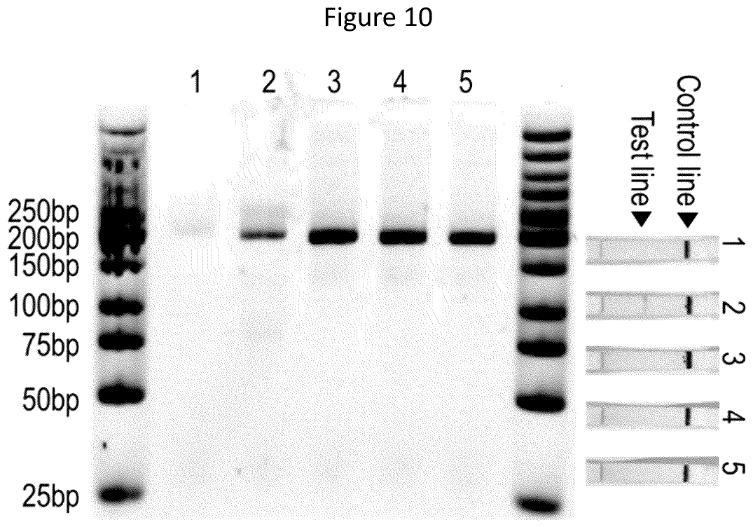

Quantity of exonuclease III: 25 μl of reaction buffer (10 mM Tris-HCL, 10 mM MgOAC, pH 7.0) containing the amplicon, 400 nM NG probe, and various quantities of exonuclease III were incubated at 38° C. for 10 minutes. After the incubation, 2 μL of the digested amplicon was loaded onto a LFA strip. The rest of the digested amplicon was purified using the Monarch® PCR & DNA Cleanup Kit and analyzed using gel electrophoresis. The results of this experiment indicate that 10 U of exonuclease III completely digested the amplicon-probe hybrid, resulting in no visible test line on the LFA strip, while 100 mU, 10 mU, and 1 mU of exonuclease III did not digest the amplicon sufficiently to generate a visible test line (FIG. 10). Hence, 1 U of exonuclease III per 25 μl reaction mixture was found to be the most suitable concentration for use in the method.

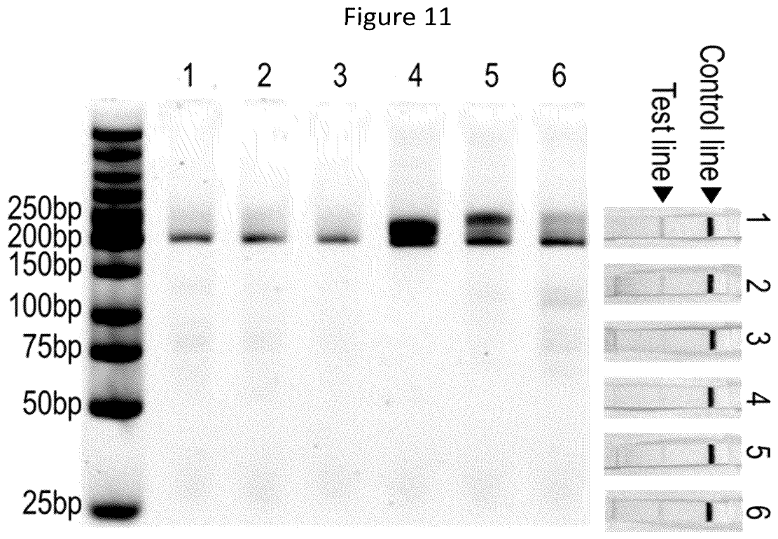

Digestion temperature and time: Using 1 U exonuclease III per 25 μL reaction, digestions were performed at room temperature (RT, 20° C.) and 38° C. for three different lengths of time (i.e., 5 minutes, 10 minutes, and 20 minutes) (FIG. 11). At 38° C., the strongest test line signal was obtained in 5 minutes. However, when this digestion was prolonged to 20 minutes, the test line signal nearly disappeared. For the incubation at RT, the test line signal intensity increased as the digestion time was increased to 20 minutes, but the intensity remained lower than that obtained by digestion at 38° C. for 5 minutes. Using gel electrophoresis, we confirmed that the digestion at RT was not complete after 20 minutes. It is likely that by using more exonuclease III, digestion can be accelerated at RT. However, at lower temperatures, probe-amplicon hybridization might be hindered by the formation of DNA secondary structures.

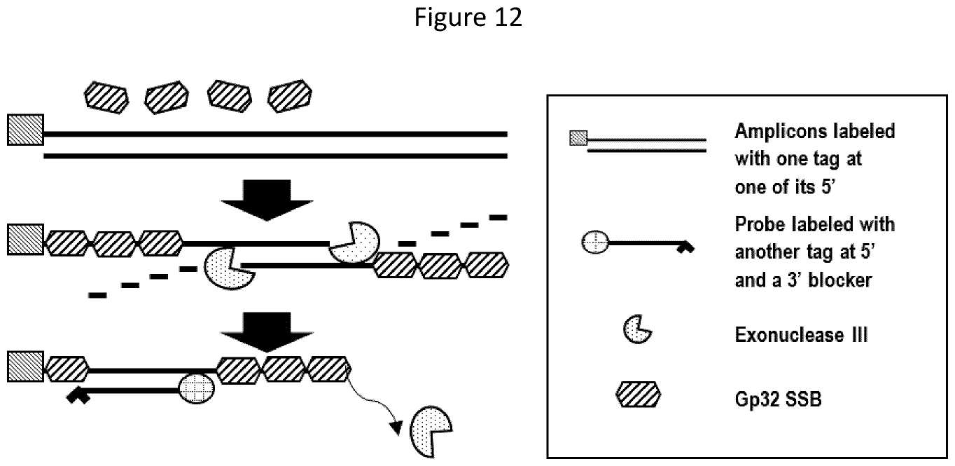

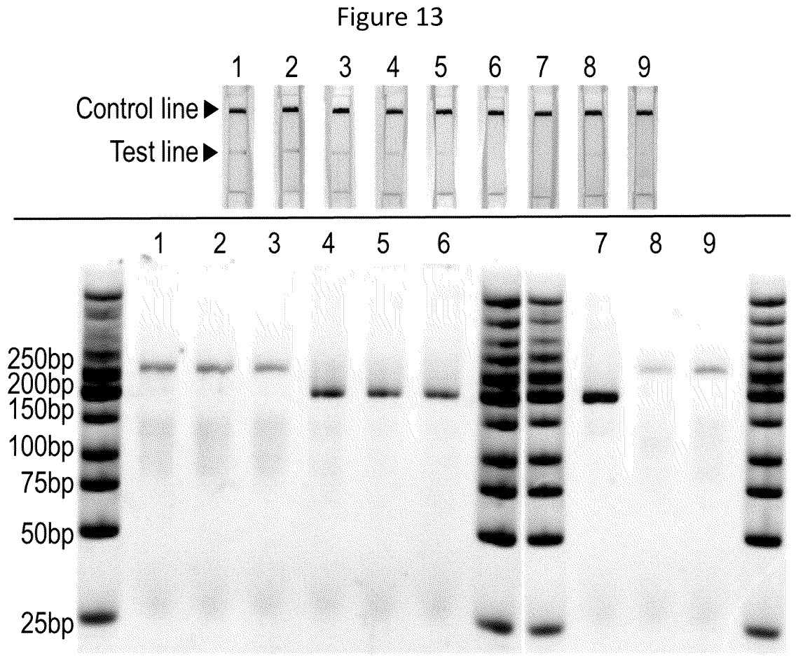

Addition of gp32: The single-stranded DNA (ssDNA) binding protein gp32 (bacteriophage T4 gene 32) binds to ssDNA and protects it from exonuclease digestion (FIG. 12). It can also accelerate the hybridization of DNA (Plos one 13(4): e0194357, 2018; Nature 227: 1313-1318, 1970). Thus, we tested the ability of 400 ng/μL gp32 (NEB, M0300S) to stabilize the amplicon-probe hybrid in the separate digestion step described above. As shown in FIG. 13, gp32 stabilizes the amplicon-probe hybrid against digestion by 1 U exonuclease III in a 40-minute incubation at 38° C. Even with 10 U exonuclease III, the LFA test line signal is still visible after a 20-minute incubation at 38° C. As in previous experiments, a concentration of 20 U exonuclease III per 25 μL reaction is optimal for use in RPA reactions that contains gp32. In the electrophoresis results, the amplicon treated with exonuclease III and gp32 appears as an upshifted band due to gp32 binding to the DNA, which suggests that the cleanup kit does not remove gp32. However, when exonuclease III is not present (i.e., in sample 7), gp32 does not bind to the amplicon and no upshifting is observed. To demonstrate that the addition of gp32 does not affect the specificity of detection, a non-specific NG probe (5′-/5Biosg/AAACGAGCCGAAATCACTGA/3InvdT/-3′ (SEQ ID NO: 13)) that targets a gene that is not homologous to the target amplicon (i.e., the porA pseudogene) was used (see reaction 9 in FIG. 13). This reaction did not generate a visible test line signal regardless of the presence of a digested amplicon. These results suggest that the use of gp32 protects the amplicon-probe hybrid from further digestion by exonuclease III in the separate digestion step and will allow the disclosed detection method to be performed on amplicons generated by other nucleic acid amplification methods.

What is claimed:

1. A method for detecting a target nucleic acid, the method comprising:
a) digesting a target amplicon comprising the target nucleic acid and a first 5′ tag with an exonuclease III;
b) hybridizing a probe to a single-stranded portion of the digested amplicon that comprises the first 5′ tag to form an amplicon-probe hybrid, wherein the probe comprises a second 5′ tag and a 3′ blocker; and
c) detecting the amplicon-probe hybrid.

2. The method of claim 1, wherein digestion of the target amplicon and hybridization of the probe occur contemporaneously in a single vessel.

3. The method of claim 1 further comprising preparing the target amplicon, wherein the target amplicon is prepared by:
a) combining a sample comprising the target nucleic acid with a forward primer comprising the first 5′ tag, a reverse primer, deoxynucleotide triphosphates (dNTPs), and an amplification master mix to form a reaction mixture, wherein the forward primer and the reverse primer are complementary to the ends of the target nucleic acid; and
b) incubating the reaction mixture under conditions suitable for amplification to form the target amplicon.

4. The method of claim 3, wherein incubation of the reaction mixture and digestion of the target amplicon occur contemporaneously in a single vessel.

5. The method of claim 4, wherein incubation of the reaction mixture, digestion of the target amplicon, and hybridization of the probe all occur contemporaneously in the vessel.

6. A method for assaying for the presence of a target nucleic acid in a sample, the method comprising:
a) combining the sample with a forward primer comprising a first 5′ tag, a reverse primer, dNTPs, and an amplification master mix to form a reaction mixture, wherein the forward primer and the reverse primer are complementary to the ends of the target nucleic acid;
b) incubating the reaction mixture under conditions suitable for amplification to form an amplification product, wherein the amplification product comprises a target amplicon comprising the target nucleic acid and the first 5′ tag if the target nucleic acid is present in the sample;
c) incubating the amplification product with an exonuclease III to form a digestion product, wherein the digestion product comprises a single-stranded digested target amplicon comprising the first 5′ tag if the target amplicon is present in the amplification product;
d) incubating the digestion product with a probe, wherein the probe hybridizes to the single-stranded digested target amplicon to form an amplicon-probe hybrid if the single-stranded digested target amplicon is present in the digestion product, and wherein the probe comprises a second 5′ tag and a 3′ blocker and is complementary to a portion of the single-stranded digested target amplicon; and
e) assaying for the presence of the amplicon-probe hybrid.

7. The method of claim 6, wherein step (c) and step (d) occur contemporaneously in a single vessel.

8. The method of claim 6, wherein step (b) and step (c) occur contemporaneously in a single vessel.

9. The method of claim 10, wherein step (b), step (c), and step (d) all occur contemporaneously in a single vessel.

10. The method of any one of claims 3-9, wherein the sample is a patient sample, an environmental sample, or a food sample.

11. The method of any one of claims 3-10, wherein the target nucleic acid is from a pathogen, and wherein detection of the amplicon-probe hybrid indicates that the pathogen is present in the sample.

12. The method of any one of claims 3-11, wherein incubation of the reaction mixture occurs under isothermal conditions.

13. The method of claim 12, wherein the amplification master mix comprises RPA reagents.

14. The method of claim 13, wherein the amplification master mix comprises a recombinase, a recombination mediator protein, a single-stranded DNA binding protein (SSB), and a stand-displacing DNA polymerase.

15. The method of claim 14, wherein the recombinase is UvsX from T4 bacteriophage.

16. The method of claim 14 or 15, wherein the recombination mediator protein is UvsY from T4 bacteriophage.

17. The method of any one of claims 14-16, wherein the SSB is gp32.

18. The method of any one of claims 14-17, wherein the stand-displacing DNA polymerase is the large fragment of Bacillus subtilis polymerase I (Bsu).

19. The method of any one of claims 12-18, wherein incubation of the reaction mixture is performed at 37-40° C. for 20-60 minutes.

20. The method of any one of claims 3-19, wherein the target nucleic acid is RNA, and wherein the reaction mixture further comprises a reverse transcriptase.

21. The method of any one of claims 1-20, wherein the exonuclease III is from E. coli.

22. The method of any one of claims 1-20, wherein the exonuclease III is thermostable.

23. The method of any one of the preceding claims, wherein the probe is 15-40 nucleotides in length.

24. The method of any one of the preceding claims, wherein the 3′ blocker is an inverted base.

25. The method of any one of the preceding claims, wherein the amplicon-probe hybrid is detected by binding the amplicon-probe hybrid to a first binding agent immobilized on a substrate and to a second binding agent conjugated to a detectable label, and wherein the first binding agent specifically binds to either the first 5′ tag or the second 5′ tag and the second binding agent specifically binds to the other 5′ tag.

26. The method of claim 25, wherein either the first 5′ tag or the second 5′ tag is fluorescein isothiocyanate (FITC), and either the first binding agent or the second binding agent is an anti-FITC antibody.

27. The method of claim 25 or 26, wherein either the first 5′ tag or the second 5′ tag is biotin, and either the first binding agent or the second binding agent is streptavidin.

28. The method of any one of claims 25-27, wherein the detectable label is a colored bead, a fluorescent bead, a gold nanoparticle, or a quantum dot.

29. A kit for detecting a target nucleic acid in a sample, the kit comprising:
a) a forward primer comprising a first 5′ tag;
b) a reverse primer;
c) a probe comprising a second 5′ tag and a 3′ blocker; and
d) an exonuclease III;
wherein the forward primer and the reverse primer are complementary to the ends of the target nucleic acid and the probe is complementary to an intervening portion of the target nucleic acid.

30. The kit of claim 29 further comprising dNTPs and/or an amplification master mix.

31. The kit of claim 29 or 30 further comprising a detection apparatus.

32. The kit of claim 31, wherein the detection apparatus is a lateral flow device.

33. A composition comprising:
a) a forward primer comprising a first 5′ tag and a reverse primer, wherein the forward primer and the reverse primer are complementary to the ends of a target nucleic acid;
b) dNTPs;
c) an amplification master mix;
d) an exonuclease III; and
e) a probe comprising a second 5′ tag and a 3′ blocker, wherein probe is complementary to the target nucleic acid.

34. A method for assaying for the presence of a target nucleic acid in a sample, the method comprising:
a) combining the sample with the composition of claim 33 to form a reaction mixture;
b) incubating the reaction mixture under conditions suitable for amplification to form a reaction product, wherein the reaction product comprises an amplicon-probe hybrid if the target nucleic acid is present in the sample, and wherein the amplicon-probe hybrid comprises the probe hybridized to a portion of a single-stranded digest of an amplicon of the target nucleic acid comprising the first 5′ tag; and
c) assaying for the presence of the amplicon-probe hybrid.

35. The composition of claim 33 further comprising:
f) the target nucleic acid;
g) an amplicon of the target nucleic acid comprising the first 5′ tag; and
h) an amplicon-probe hybrid, wherein the amplicon-probe hybrid comprises the probe hybridized to a portion of a single-stranded digest of an amplicon of the target nucleic acid comprising the first 5′ tag.


SEQUENCE LISTING (13 sequences)

SEQ ID NO: 1 - DNA, 248 bp, Neisseria gonorrhoeae
  source 1..248: mol_type = genomic DNA
tattgatgcgaatcaagaccagccatacagcaacgaactatatgaagcattgcagcaaaacattgcccaagattccgttatggcttcgcagagaacaagcaaggccatgaatttggcctcggttcgtaaagctattaatcaatttgtaaaaatgggatttatagaaccgtttttatcaagctatacacctttatcacgagattatgtacaagctcgtactaaccgaaaacgacaaaccctactttcca

SEQ ID NO: 2 - DNA, 28 bp, synthetic construct
  source 1..28: mol_type = other DNA
gattccgttatggcttcgcagagaacaa

SEQ ID NO: 3 - DNA, 30 bp, synthetic construct
  source 1..30: mol_type = other DNA
tggaaagtagggtttgtcgttttcggttag

SEQ ID NO: 4 - DNA, 18 bp, synthetic construct
  source 1..18: mol_type = other DNA
ccaaattcatggccttgc

SEQ ID NO: 5 - DNA, 29 bp, synthetic construct
  source 1..29: mol_type = other DNA
ctatagttgaatcttctaccactgaagca

SEQ ID NO: 6 - DNA, 29 bp, synthetic construct
  source 1..29: mol_type = other DNA
gtaggtactgtaggtttattgatttgaga

SEQ ID NO: 7 - DNA, 29 bp, synthetic construct
  source 1..29: mol_type = other DNA
gtggtgctaaggcaatgatagaagatgga

SEQ ID NO: 8 - DNA, 32 bp, synthetic construct
  source 1..32: mol_type = other DNA
gcacttccatgaccacctcttccaataacttc

SEQ ID NO: 9 - DNA, 20 bp, synthetic construct
  source 1..20: mol_type = other DNA
ggcatattgtgccatccaaa

SEQ ID NO: 10 - DNA, 30 bp, synthetic construct
  source 1..30: mol_type = other DNA
atcaaccagataggtaggtaatggaatgac

SEQ ID NO: 11 - DNA, 28 bp, synthetic construct
  source 1..28: mol_type = other DNA
ctacaagcatgaaatggcagaacagcgt

SEQ ID NO: 12 - DNA, 20 bp, synthetic construct
  source 1..20: mol_type = other DNA
ggtactaatggtgatgatca

SEQ ID NO: 13 - DNA, 20 bp, synthetic construct
  source 1..20: mol_type = other DNA
aaacgagccgaaatcactga